(19) United States
(12) Patent Application Publication
(10) Pub. No.: US 2005/0090718 A1
(43) Pub. Date: Apr. 28, 2005

(54) Animal healthcare well-being and nutrition

(76) Inventor: W Jean Dodds, Santa Monica, CA (US)

Correspondence Address: FULBRIGHT & JAWORSKI, LLP, 1301 MCKINNEY, SUITE 5100, HOUSTON, TX, 77010-3095, US

(21) Appl. No.: 10/481,508

(22) PCT Filed: Jul. 3, 2002
(86) PCT No.: PCT/US02/21147

Related U.S. Application Data
(63) Continuation-in-part of application No. 09/898,193, filed on Jul. 2, 2001, now Pat. No. 6,537,213.
Application No. 09/898,193 is a continuation-in-part of application No. 09/432,851, filed on Nov. 2, 1999, now Pat. No. 6,287,254.
(60) Provisional application No. 60/305,443, filed on Jul. 13, 2001.

(51) Int. Cl. A61B 5/00

(52) U.S. Cl. 600/300

(57) ABSTRACT

This invention relates to a method, system and apparatus for the management of comprehensive and cumulative genetic and health assessment databases in relation to animals worldwide. More specifically, the invention is directed to methods and systems that are used to determine animal health care, well-being and nutrition.

This application relates to application Ser. No. 09/419,192, which was filed on Oct. 15, 1999; application Ser. No. 09/432,851, which was filed on Nov. 2, 1999; and application Ser. No. 09/898,193, which was filed on Jul. 2, 2001; and Application Ser. No. 60/305,443, which was filed Jul. 13, 2001. The contents of these applications are incorporated by reference herein.

### BACKGROUND OF THE INVENTION

A. Technical Field

This invention relates to a method, system and apparatus for the management of comprehensive and cumulative genetic and health assessment databases in relation to animals worldwide. In particular, the invention relates to a bioinformatics system and its implementation in relation to animal biological data. More specifically, the invention is directed to methods and systems that are used to determine animal health care, well-being and nutrition.

B. Related Art

Substantial investments in time, effort and financial resources are made by breeders, owners, and caregivers of animals, particularly purebred animals, to characterize the animal's health state. This characterization may include determining the animal's genetic background and predicting the animal's morbidity, mortality and longevity. The probability that an individual animal will develop a specific health-related condition in its lifetime is a product of complex interactions between its genetic makeup, environmental influences including diet, and agents of disease (i.e., chemical, physical, or biological) that it encounters. Thus, perhaps the best indicator of overall health of an individual animal or breed is longevity.

1. Phenotype Data

Generally, the phenotype is the genetic nature of an organism that is revealed by visible characteristics or measurable performance. Phenotype data or information includes physical descriptive and health assessment profile characteristics. These characteristics, include, for example physiological, pathological, endocrinological, hematological, epidemiological, behavioral, and immunological data from parameters such as phenotype, breed, lifespan, health history, and presence of infectious diseases and metabolic disorders.

Blood and other biological samples of a subject animal are analyzed by laboratories having a central database processing resource (CDPR). This is a system for obtaining the phenotypic information. Communication systems are known for connecting these laboratories with veterinary clinics through a telephone and/or fax connection on an automated basis. These systems permit the veterinarian, animal hospital, or other authorized person (collectively or individually termed the “remote user”) to receive the health assessment profile and basic descriptive identifying data, namely phenotypic information, of a subject animal from the CDPR. Until recently, it was not possible for the remote user to access the CDPR directly to obtain this phenotypic information of a subject animal.

Typically, a breeder and/or owner of animals obtains health assessments of their animals by submitting blood or other body fluid or tissue samples to a veterinarian or veterinary clinic. The veterinarian or veterinary clinic submits the sample to a laboratory for analysis of the biological, physiological, or pathological condition of the animal. The data (physical health of the animal) are reported to the owner through the veterinarian or veterinary clinic. The data also can be stored on the CDPR of the laboratory. Additionally, for each subject animal, the phenotypic data can be stored on a computer storage system at the veterinary clinic or in a computer storage system of the owner and/or breeder. The retrieval of the data can be electronically, by voice, hard copy, or fax as required.

Seeking, obtaining and storing this phenotypic information is driven by the needs of the animal breeder, owner or the agent of the owner and the animal's healthcare provider. This information is sought to resolve the clinical, diagnostic, management, and therapeutic needs of an animal subject when the animal is in need of periodic wellness examination, is ill, or is to be restored to a well condition.

2. Genotype Data

A second aspect of data associated with animals is genetic or genotype data or information. The genetic constitution of an organism includes genes without visible effects as well as those revealed by the phenotype. Genetic data are typically used to estimate the presence and prevalence of disease and/or disorder among different breeds or kinds of animals. Genotypic information is most often stored manually in a non-CDPR facility, for example, select clinical research databases, book form, hard copy, or genetic disease registries.

Some of the genetic registries are related to specific diseases or disorders, for instance, hip dysplasia, eye conditions, thyroid conditions, and blood conditions. When retained in a genetic disease registry, the data typically list only those animals that are not affected with or carrying the heritable trait in question. These genetic registries are normally the subject of confidential knowledge of a breeder and/or owner, and not the subject of a generally accessible database. These are retained as confidential by the owners either for financial reasons, risk reasons, legal liability reasons, or personal reasons.

Thus, genotypic information is transmitted manually to and from persons or local and national genotypic databases maintained for specific disorders, and designed to foster research into diseases and disorders, rather than being readily accessible to users for clinical purposes in the manner of phenotypic data on a CDPR

3. Failings of the Existing Systems

There is a need to develop these data in a cumulative, comprehensive, and dynamic system of database management to thereby enhance the health predictability, and longevity of animals. This type of comprehensive and cumulative database on individual or groups of animals needs to be preserved and shared locally, regionally, nationally, and globally. A mechanism to do this is presently not known due to the various constraints surrounding each of the two types of databases. The phenotype database storage, use, and access is fashioned, formed and structured for use by clinical laboratories and veterinarians. The genotype information is fashioned and structured generally for clinical research and breeder/owner uses as opposed to clinical medical uses.

There is also a need for a new database management bioinformatics scheme and relational database, together with computerized networks that manage, analyze, and/or integrate comprehensive and cumulative animal health assessment data and genetic identifier, genomic mapping, and genetic assessment data. Current laboratory and research systems and computerization have not achieved this, and nor have communication protocols been used effectively in this technological area to facilitate such a relationship or relational bioinformatics database system for management and dissemination of this comprehensive and cumulative information.

More specifically, it is necessary in animal health diagnosis and care that appropriate predictive testing for diseases and disorders of animals be achieved in order to reduce morbidity and mortality, and improve the quality of life and lifespan. Currently, available testing is unnecessarily complex and expensive in relation to the ability to be an accurate predictor of diseases and disorders in animals.

The present invention is the first to store and/or present phenotypic information and genotypic information as a comprehensive and cumulative assessment of individual animal subjects, families of subjects, breeds of subjects, or species of animals in a computerized format which is available through computer networking to authorized remote users.

### BRIEF SUMMARY OF THE INVENTION

One embodiment of the present invention is a dynamic method and system of managing the health care and well-being of a non-livestock animal subject. Such an animal is preferably a canine subject or a feline subject. This method comprises obtaining a database relating to at least one of the species of the animal or selected group of the species. Next, the method comprises obtaining data relating to the animal, for example laboratory test data relating to the animal. The next step involves relating the database with the data using a computer; and determining a regimen for the management of the animal.

A further embodiment of the present invention is a method of managing the nutrition of a non-livestock animal. This method comprises obtaining a database relating to at least one of the species of the animal or selected group of the species. Next, the method comprises obtaining data relating to the animal, for example laboratory test data relating to the animal. The next step involves relating the database with the data using a computer; and determining a nutritional regimen for the management of the animal. The nutritional regimen is at least related to the nutrient or caloric composition, or the food allergies and food intolerances. The therapeutic intervention or maintenance may include drugs, nutraceuticals, vitamins, antioxidants, holistic treatments, exercise or liquid intake.

Another embodiment of the present invention is a method of managing the health of a non-livestock animal. This method comprises obtaining a database relating to at least one of the species of the animal or selected group of the species. Next, the method comprises obtaining data relating to the animal, for example laboratory test data relating to the subject. The next step involves relating the database with the data using a computer; and determining a therapeutic intervention or maintenance for the management of the animal.

The database of at least one of the species or the group is periodically updated thereby to obtain cumulative data of the species or group. Preferably both these data bases are used, and preferably both are updated to obtain the cumulative data. The data of the animal is periodically updated thereby to obtain cumulative data. Preferably, both the databases are periodically updated. The updating picks up data drift in different populations of the animals, groups and species over time, and thereby allows for the regulation of the database so as to be substantially or essentially current.

The invention also includes the step of reporting the determination of the health care, well-being, nutrition or other therapeutic requirements and suggestions or health on a communications network including the Internet. Preferably, there is a payment procedure for the report which is achieved through the Internet. This communication network and structure is described here in further detail.

Yet further, another embodiment of the present invention is a method of modulating disease of a non-livestock animal. This method comprises obtaining a database relating to at least one of the species of the animal or selected group of the species. Next, the method comprises obtaining genotypic data relating to the animal, for example genotypic data relating to the animal. The next step involves relating the database with the data using a computer; and determining a nutritional regimen to modulate the disease.

Another embodiment of the present invention is a method of modulating a disease in a non-livestock animal comprising performing a DNA test to determine a known DNA marker associated with the disease and determining a nutritional regimen to modulate the disease based on the data obtained from the DNA test.

The foregoing has outlined rather broadly the features and technical advantages of the present invention in order that the following detailed description of the invention may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is an overall view of a web-based system to provide access to a database management system of an animal genetic database and a health assessment database of the invention, in relation to the Internet.

FIG. 2 is a graphical illustration of a computer network, namely the Internet.

FIG. 3 is a block diagram of an exemplary computer system for practicing various aspects of the invention.

FIG. 4 is a view of a browser for the database management system for accessing an animal genetic database and a health assessment database of the invention.

FIG. 5 is a basic flow diagram illustrating an exemplary process by which an operator of a CDPR receives and transmits data relating to health assessment and genetic information.

FIG. 6 is a detailed flow diagram of the system steps employed in one embodiment of the present invention wherein a remote user accesses and outputs data.

FIG. 7 is a detailed flow diagram of the methods and steps employed by a remote user to add data to the database.

FIG. 8 is a flow chart illustrating an exemplary process by which the laboratory dynamically contributes, transmits and receives data associated with health assessment and genetic data to the CDPR

### DETAILED DESCRIPTION OF THE INVENTION

The present invention is now described in detail with reference to a few preferred embodiments thereof, as illustrated in the accompanying drawings. In the following description, numerous specific details are set forth in order to provide a thorough understanding of the present invention. It is apparent, however, to one skilled in the art, that the present invention may be practiced without some or all of these specific details. In other instances, well known process steps have not been described in detail in order to not unnecessarily obscure the present invention.

As used herein, the use of the word “a” or “an” when used in conjunction with the term “comprising” in the sentences and/or the specification may mean “one,” but it is also consistent with the meaning of “one or more,” “at least one,” and “one or more than one.”

As used herein, the term “companion animal” includes, but not limited to dogs, cats, horses, farm animals, food animals, live-stock animals, zoo animals or wild animals. Preferably, specific families of companion animals include, but are not limited to canine, feline, equine, bovine, porcine, avian, caprine, and ovine. More preferably, companion animals include canine species, feline species, avian species and equine species.

The term “disease” as used herein is defined as a disorder or condition that disrupts body functions, systems or organs.

As used herein, the term “genotypic information” refers to information relating to the genetic constitution of an animal. This information may include data obtained from the pedigree, family history, heritable physical characteristics, genetic screening tests, DNA testing, genomic mapping, and related laboratory assessment of the gene product for known or suspected congenital and heritable traits of a particular animal, animal family, line, or group of animals.

As used herein, the term “gene product” refers to the specific phenotypic characteristic(s) resulting from the expression of the genotype, and may include certain specific laboratory test data.

As used herein, the term “group” has many different characteristics. It can include, for example, a specific breed of animal, a specific purpose for which these animals are used, such as those who are purely companion pets in a home situation, performance animals for show conformation, for obedience, working trials, coursing trials, and for sheep herding and other herding purposes. It can also involve groups of animals depending on where they live—in a temperate climate, a warm or tropical climate, an arid desert climate, or a cold northern climate. It can include, of course, animals that live in urban and rural areas, animals that live near water, animals of various ages, intact or neutered, and for reproduction. In other words, the term “group” is used in a very broad sense and can apply to any group that the user wishes to inquire of the database. Thus, the term group is any selected subset of the healthy or diseased or disordered animals within the entire database.

As used herein, the term “health assessment profile of an animal” typically relates to a particular subject of the group, as opposed to the group of animals as a whole.

As used herein, the term “heterozygotes” refers to having different allelic genes at one or more paired loci in homologous chromosomes. One of skill in the art is aware that a heterozygote animal is an animal that contains one allele for a specific disease or is missing an allele resulting in a specific disease. Thus, a heterozygote animal is typically referred to as a carrier for the disease.

As used herein, the term “nutraceutical” compositions are preparations of natural ingredients that are multi-component systems consisting of preferably synergistic natural products and supplements to promote good health. Nutraceutical compounds can be derived from medicinal plants.

As used herein, the term “nutritional supplements” refers to nutraceuticals, anti-oxidants, vitamins, minerals, or nutraceuticals.

As used herein, the term “nutritional regimen” may include, but is not limited to pre-selected, pre-prepared food, foostuffs, treats, drinks, nutritional supplements, holistic treatments or exercise.

As used herein, the term “phenotypic information” refers to physical attributes, breed and other descriptive and health assessment information. Thus, one of skill in the art is aware that a phenotype is assigned on the basis of one or more characteristics that are observable clinically or by laboratory means that reflect genetic variation or gene-environment interaction.

The term “prophylactic” as used herein is defined as a drug or agent which acts to prevent a disease or condition.

The term “treatment” as used herein is defined as the use of a drug as a therapeutic or prophylactic therapy. A skilled artisan is cognizant that “treatment” refers to the management and care of an animal for the purpose of combating a disease, disorder or condition. Thus, the term treatment as used herein is all inclusive of the acts of curing a disease, preventing a disease or merely managing a disease.

The term “therapeutic” as used herein is defined as a drug or agent, which acts to treat a disease or condition.

A. Genetic Screening and Counseling of Purebred Animals

The common practice to line-breed and inbreed purebred animals facilitates the transmission and recognition of congenital and heritable defects. Large-scale screening programs for the identification of genetically affected and carrier animals are an effective way to discover and eventually control the frequency of these defects within the population at large. Screening programs of this type have been used successfully in humans for many years (i.e., Tay-Sachs disease, phenylketonuria) and more recently have been applied to animals (i.e., mannosidosis in cattle; hip dysplasia, eye, blood and heart diseases in dogs). Genetic screening may be essential to the survival of breeds in which mild or moderately severe defects have been propagated unknowingly for many generations.

Most purebred animals raised today have evolved over the years from a relatively small gene pool. Even though a particular genetic disorder may initially have been recognized in a specific line or family within a breed, all important breeding stock of the breed need to be screened because their genotype evolved from the original restricted gene pool. If this approach is not taken, the frequency of genetic defects in the breed will inevitably increase and have a negative impact on overall health and longevity.

Depending on the mode of inheritance, different approaches may need to be applied for the detection and control of genetic disorders. It is advantageous to be able to select against heterozygotes (carriers) rather than have to eliminate affected individuals from a breeding program once the condition is manifested. Control and elimination of the disease by testing are feasible and reliable in cases where the asymptomatic or carrier state has an expressed phenotypic and/or biochemical marker (i.e., as measured in a blood, urine or saliva test, electrocardiogram, skin biopsy, eye examination, or hair analysis). Some current examples include testing for bleeding disorders like von Willebrand disease and hemophilia; autoimmune thyroid disease leading to hypothyroidism; various eye, heart, metabolic enzyme and storage disorders; and bone and neuromuscular diseases.

B. Features Related to Genetic and Other Data Associated with Animals

1. Physical Characteristics of Disease

In the early days when animal breeders began recognizing recurring symptoms of disease states or physical characteristics, the undesirable features of these traits led them to select away from the problems by test mating and eliminating affected animals from the breeding pool. While this remains one way to select against inherited and congenital diseases, more reliable approaches have been implemented by screening for biochemical markers and most recently by using molecular genetic techniques.

A comprehensive worldwide database contains the following information for individual purebred animals:
Host characteristics: age, sex, neuter status, pedigree, height, weight, body mass index, coloration and markings, eye color, etc.
Diet: type and amount of dog and human foods consumed, vitamin and mineral supplements, frequency of feeding. This is used to derive the percentage of calories derived from fat, carbohydrate, and protein.
Medical history: occurrence of diseases, infections, etc., including date of onset, treatment, duration, and outcome, cause of death and method of diagnosis; type and amount of medications used for treatment or prevention of disease; type and frequency of vaccinations.
Personality and temperament: based on previously used personality scales.
Laboratory data: consists of routinely collected blood, serum chemistry tests, urinalysis, etc., as well as laboratory tests performed to screen for or diagnose specific conditions such as immune-mediated thyroiditis, hypothyroidism, cancer, etc.
Special diagnostic test results: include tests for hip dysplasia, congenital eye diseases, congenital heart diseases, blood disorders, and other suspected inherited disorders as tests become available.
Genetic information: derived from the canine genome project as well as tests for specific inherited conditions such as progressive retinal atrophy, hemophilia, and von Willebrand disease.

Although this comprehensive worldwide database is available for individual purebred animals, there still exists a need to develop a database for none purebred animals. Thus, the present invention contemplates the development of such a database containing phenotypic and genotypic information relating to purebred and non-purebred animals. It is envisioned that this database can be used to predict and/or diagnosis disease in an owner's and/or breeder's animal. Yet further, the present database can be used to manage diseases and/or prevent diseases by predicting the disease and determining a nutritional regimen for management and/or prevention of a disease in the animal.

2. Phenotypic Markers of Disease

For about three decades, veterinary and comparative geneticists have developed and relied upon physical and biochemical markers of specific genetic traits to identify carrier (heterozygotes) and affected animals. These methods aimed to produce reliable, practical, and affordable tests that may be predictive of the gene product, and therefore the genotype of a particular genetic disorder. To be considered accurate and predictive, retrospective analyses of data developed from these testing programs were compared to the pedigrees of animals being screened as a means of validating the tests. Such genetic screening tests may be considered reliable if they correctly identified animals as having the normal and abnormal genotypes at least 80% of the time.

An important indicator of overall health of an individual animal or breed is longevity. Relationships between a specific health-related condition and an animal's genetic, environmental influences and lifespan have been characterized, in part, for several important diseases of dogs including bone cancer (osteosarcoma) and gastric dilatation-volvulus (GDV). For example, the risk of osteosarcoma increases with increasing age, increasing weight and increasing height. Compared with the German shepherd breed, the highest risk of osteosarcoma occurs among large and giant breeds, while small breeds have reduced risk. Furthermore, the risk of osteosarcoma is increased two-fold in neutered dogs. Yet further, factors that increase the risk of GDV in purebred dogs are male gender, being underweight, eating only one meal per day, eating rapidly, and a fearful temperament. Factors that decrease the risk of GDV include a happy temperament and inclusion of table foods in the diet. The lifetime risk of developing GDV in large and giant breed dogs is 20% and 23%, respectively, whereas the lifetime risk of dying of GDV for these breeds is 6%.

Similarly, the comparative longevity of different dog breeds has been described using the age of death and other descriptive characteristics of more than 38,000 dogs that were included in a large veterinary database. Predictable relationships were found between the breed and size of dogs and the average age of death. It was noted that dogs are unique among animal species in having a more than 50-fold difference in adult body size and a corresponding large difference in longevity between the smallest and biggest dog breeds. Since these dog breeds have more than 99% of their genome in common, it suggests that the genetic code for both size and longevity is contained within a very small part of the dog's genome. As mapping of the canine genome progresses, it is possible to identify not only genes that code for specific diseases such as cancer and GDV, but also for the genes that determine body size and longevity.

3. Genotypic Markers of Disease

Recent advances in molecular genetics have focused on mapping the human genome, and this has stimulated interest in developing parallel genetic maps for animals. For example, it is estimated that a minimum of ten years and several million dollars is needed to map the canine genome. Once developed, a genetic map provides information about the relative order and placement of genes or specific DNA markers on specific chromosomes. This allows one to locate specific regions on chromosomes where genes of interest are likely to be found. Once a molecular marker is identified close to a specific gene of interest, screening tests for this particular marker can be used to identify individuals carrying or expressing the trait.

Thus, the present invention has contemplated the use of a single test, such as a DNA test, to determine the genetic profile of the animal. Once a DNA marker is determined for a specific disease and/or disorder, a standard DNA test well known and used by those of skill in the art can be preformed to determine the presence and/or absence of the DNA marker. Such test may include, PCR-based methods, Southern blot, oligonucleotide arrays, etc.

Some of the characteristics of animals with which this invention is concerned are the following:

| | | | Genotype & |
|---|---|---|---|
| | | | Some |
| | Mostly | | Phenotype |
| Animal Characteristics | Phenotype | Mostly Genotype | (Gene Product) |
| Species | | | X |
| Purebred | | | X |
| Crossbred | X | | |
| Mixed breed | X | | |
| Size | X | | |
| Weight | X | | |
| Age | X | | |
| Sex | | | X |
| Lifespan | | | X |
| Body type | X | | |
| Color | | | X |
| Family history | | X | |
| DNA testing | | X | |
| Genomic mapping | | X | |
| Blood type | | X | |
| Thyroid function | X | | |
| von Willebrand factor | | | X |
| Hemophilia | | | X |
| Other bleeding disorders | X | | |
| Glucose | X | | |
| Cholesterol | X | | |
| Alkaline phosphatase | X | | |
| Alanine aminotransferase | X | | |
| Bile acids | X | | |
| Cortisol | X | | |
| Cataracts | X | | |
| Progressive retinal | | | X |
| atrophy | | | |
| Microophthalmia | | | X |
| Dry eye (KCS) | X | | |
| Hip dysplasia | X | | |
| Arthritis | X | | |
| Temperament | X | | |
| Ruptured cruciate | X | | |
| ligament | | | |
| Hemolytic anemia | X | | |
| Urinalysis | X | | |
| Kidney stones | | | X |
| Bloat (gastric dilatation) | | | X |
| Pyoderma | X | | |
| Seborrhea | X | | |
| Sebaceous adenitis | | | X |
| Umbilical hernia | X | | |
| Inguinal hernia | X | | |
| Epilepsy | | | X |
| Heartworm disease | X | | |
| Cardiomyopathy | X | | |
| Patent ductus arteriosus | | | X |
| Immunoglobulin levels | X | | |

In the category of genotype and some phenotype, the phenotype component (measurable gene product) is typically less than 20%.

C. Diagnostic Testing

The development of one or more assays or techniques for performing the invented testing protocols, standards and procedures of the present invention is straightforward, and within the knowledge of a person skilled in the art. The contents of U.S. Pat. No. 5,830,7009 (Benson) entitled “Detection Method for Homologous Portions of a Class of Substances” is indicative of some of the tests and formats that are possible. The contents of that patent are incorporated by reference herein.

One or more of a panel of tests relate to at least one of endocrine function, immunologic function, gastrointestinal function and nutritional analysis, inborn errors of metabolism, paternity, DNA fingerprinting, hemostasis and coagulation function, vaccinal antibody status, adverse and potential adverse vaccine reaction, infectious diseases, pathology, blood typing and bone marrow analysis, cell cytotoxicity, cytokines and allergy testing, and markers of neoplastic or paraneoplastic change. These data are relevant to the likely morbidity, likely longevity, and/or the potential risk for disease or disorder for the animal. One of skill in the art is aware that as used herein the term “DNA fingerprinting” refers to genetic profiling, for example, but not limited to profiling for disease susceptibility.

The following are some specific diagnostic test panels and specialized diagnostic tests and test groups used to monitor health, morbidity, mortality and longevity of animals and animal families, and to predict the potential risks of disease or disorder.

1. Test 1: Comprehensive Diagnostic Test Panel

Patient phenotypic descriptors and genotypic descriptors/background; complete blood count (CBC) and platelet count, platelet size, platelet morphology; serum chemistry profile [i.e., AST (SGOT), ALT (SGOT), bilirubin (total, direct and indirect), alkaline phosphatase, GGT (GGTP), total protein, albumin, globulin, A/G ratio, cholesterol, BUN, creatinine, BUN/creatinine ratio, phosphorus, calcium, corrected calcium, calcium/phosphorus ratio, glucose, amylase, lipase, sodium, potassium, Na/K ratio, chloride, CPK, triglyceride, osmolality]; complete thyroid profile (total T4, total T3, free T4 (ED or other), free T3, T3 autoantibody, T4 autoantibody, TSH, thyroglobulin autoantibody); and urinalysis, urine culture, and sensitivity, if indicated.

2. Test 2: Diagnostic Test Panels for Endocrine Function

Patient phenotypic descriptors and genotypic descriptors/background, plus any or all of selected tests from the following list:
1) Thyroid Function: total T4, total T3, free T4 (ED or other), free T3, T3 autoantibody, T4 autoantibody. Molecular screening for autoimmune thyroiditis including immunoglobulin receptors on B-cells, T-cell receptors, and major histocompatibilty complex (MHC) genes Class I and II allellic HLA, DLA, or equivalent animal antigenic specificities (RFLP, PCR/SSO, PCR/SSP).
2) Adrenal Function: cortisol (basal and after stimulation with ACTH, or serially after suppression with high or low-dose dexamethazone); endogenous cortisol; and endogenous ACTH.
3) Reproductive Function: testosterone; estradiol-17β; relaxin (pregnancy diagnosis); progesterone; luteinizing hormone; estrone sulfate; follicle stimulating hormone; vaginal cytology and/or culture; testicular cytology or biopsy; prostatic cytology, biopsy or wash; screens for ovarian or testicular remnants.
4) Pancreatic Function: amylase; lipase; glucose; glucagon, trypsin-like immunoreactivity (TLI); insulin, fructosamine; glycosylated hemoglobin.
5) Parathyroid Hormone Function: parathormone; ionized calcium.
6) Other Endocrine Function: aldosterone; 21 adrenal hydroxylase; vanylla mandelic acid (VMA, for epinephrine and norepinephrine metabolities).

3. Test 3: Diagnostic Test Panels for Immunologic Function

Patient phenotypic descriptors and genotypic descriptors/background, plus any or all of selected tests from the following list:

Antinuclear antibody (ANA)—if positive, run double stranded, single stranded, speckled, anti-RNA levels; Coombs' testing (direct and indirect; elution or microbeads gel-test); rheumatoid factor; serum electrophoresis—if abnormal, run immunoelectrophoresis, isoelectric focusing, immunoblotting (Western, Northern, Southern blots); immunoglobulin levels (IgG, IgA, IgM, IgD and IgE); complement levels (C1, C1a, C1 esterase inhibitor, C3, C4, C5-C9); LE-prep testing; lupus anticoagulant (dilute Russell's viper venom test or dilutional inhibitor test); urine protein SDS-gel electrophoresis; fibronectin and anti-fibronectin antibody; flow cytometry with fluorescence activated cell sorter (FACS, for leukocyte subsets and markers such as CD4+ and CD8+; leukocyte chemotaxis (leukocyte migration inhibition test, leukotrienes); cytokines including lymphokines and monokines (macrophage-derived) such as the interleukins (IL) [i.e., IL-6 regulated by estradiol-17β, IL-8 acts as neutrophil chemotactic factor], interferons, tumor necrosis factor(s), leukotrienes, colony stimulating facors, transforming growth factor-beta and chemokines (inflammatory cytokines); anti-platelet antibody tests (serum, bone marrow); anti-megakaryocyte antibody tests (IFA, elution); and anti-leukocyte antibody tests (direct and indirect anti-neutrophil cytoplasmic antibody, antilymphocyte antibody, etc.).

4. Test 4: Diagnostic Test Panels for Gastrointestinal Function and Nutritional Analysis

Patient phenotypic descriptors and genotypic descriptors/background, plus nutritional and food supplement past and current use, plus any or all of selected tests from the following list:

Serum nutrients and vitamin analysis; CBC as in Test 1; serum chemistry as in Test 1 plus magnesium and iron; urinalysis, urine culture and sensitivity, if indicated; urine fractional excretion; serum and urine amino acid analyses; serum cobalamin (vitamin B12) and folate analysis; TLI [same as Test 2, 4)]; fecal flotation; Giardia screen, Clostridium perfringens enterotoxin test; cryptosporidiosis test (FA); toxoplasmosis test; bile acids test (resting and post-prandial); fecal alpha-1 protease inhibitor activity. If any abnormalities are present, further investigation includes ion-coupled plasma emission spectroscopy (ICP) for mineral analysis, and electrophoresis.

5. Test 5: Diagnostic Test Panels for Inborn Errors of Metabolism

Characteristics related to presence of or susceptibility to mammary cancer of the animal are determined. Biological laboratory test data from a bodily fluid or tissue of an animal are analyzed. The test data relate to estrogen (estradiol-17β), estrogen receptors, interleukin (IL) 6, progesterone, and progesterone receptors. The value should fall within predetermined levels as a determinant of presence or susceptibility to mammary cancer.

Patient phenotypic descriptors and genotypic descriptors/background, plus any or all selected tests from the following list:

Genetic screening tests including blood and urine analyses for mucopolysaccharides, cerebrosides, glycogen-storage diseases, phenylketones, phosphofructokinase, mannosidases, combined and specific immunoglobulin deficiencies/dysfunctions; skin and tissue biopsies; karyotyping for genotype determination; and DNA marker analyses.

6. Test 6: Diagnostic Test Panels for Paternity Testing and DNA Fingerprinting

Patient phenotypic descriptors and genotypic descriptors/background, plus any or all selected tests from the following list:

Major histocompatibilty complex (MHC) Class I and II alleles [analyses of HLA, DLA, or equivalent animal antigenic specificities]; genotyping; gene mapping and fingerprinting.

7. Test 7: Diagnostic Test Panels for Hemostatic and Coagulation Function

Patient phenotypic descriptors and genotypic descriptors/background, plus any or all selected tests from the following list:

Platelet count, platelet size (blood slide, mean platelet volume), platelet morphology (light, scanning, and electron microscopy); prothrombin time; partial thromboplastin time; fibrinogen; fibrin-fibrinogen degradation products (D-dimer test); platelet function tests (aggregation, release, clot retraction, whole blood aggregation, ristocetin cofactor); von Willebrand factor antigen and multimer analysis; specific coagulation factor analyses (factors II, V, VII, VIII:C, IX, X, XI, XII, XIII); fibrinolytic tests (plasminogen, plasmin, antiplasmin, tissue plasminogen activator, dilute whole blood lysis test, euglobulin lysis test); anti-thrombin III test; circulating anticoagulant tests; platelet factors 3 and 4 (heparin cofactor); protein C; protein S; kinin-kinogen tests; prekallikrein test; alpha1-antitrypsin assay; alpha2-macroglobulin assay; C1 esterase inactivator assay; anti-platelet antibody, and anti-megakaryocyte antibody tests (see Test 3).

8. Test 8: Diagnostic Test Panels for Vaccinal Antibody Status, and Adverse Vaccine or Potential Adverse Vaccine Reaction

Patient phenotypic descriptors and genotypic descriptors/background, plus any or all selected tests from the following list:
1) Serology for Vaccinal Antibody: canine distemper, canine parvovirus, canine coronavirus, canine parainfluenza virus, infectious canine hepatitis virus, canine bordetella, canine Lyme (borrelia), canine leptospirosis, rabies virus, feline panleukopenia virus, feline leukemia virus, feline infectious peritonitis virus, feline immunodeficiency virus, feline calicivirus, feline herpes virus, and equine herpes viruses (I-IV), etc.
2) Adverse Vaccine Reaction: Same as Test 3, but especially CBC; ANA; Coombs' test; platelet count, size, and morphology; anti-neutrophil cytoplasmic antibody, marker for vasculitis; complement tests; leukocyte chemotaxis tests; urine protein/creatinine ratio; anti-platelet antibody; immunoglobulin levels, especially IgG, IgA, IgM; flow cytometry (FACS) leukocyte subsets; cell cytotoxicity analysis; cytokines, especially chemokines; and complete thyroid autoantibody panel.
3) Potential (High Risk) Vaccine Reaction: especially for breeds such as the Akita, Weimaraner, Standard poodle, Eskimo Dog, harlequin Great Dane; CBC; ANA; platelet count, size and morphology; complete thyroid autoantibody panel; cell cytotoxicity analysis; cytokines; and immunoglobulin levels, especially IgG, IgA, IgM.

9. Test 9: Diagnostic Test Panels for Infectious Diseases

Patient phenotypic descriptors and genotypic descriptors/background, plus any or all selected tests from the following list:
1) North America: Ehrlichia species (E. canis, E. risticii, E. equi, E. platys, etc.); Rickettsia rickettsei (RMSF); Borrelia species (Lyme disease); Bartonella species (B. henselae, B. vinsonii, B. clarridgeiae, B. kochlerae); systemic fungal diseases (Coccidioides spp, Cryptococcus spp, Histoplasma spp, Blastomyces spp, Aspergillus spp, ringworm); mange mites (Demodex, Sarcoptes, Chyletiella, etc.); enteric diseases (Clostridium perfringens enterotoxin); protozoan diseases (Toxoplasma spp.; Coccidia spp; Giardia spp); retrovirses (feline leukemia virus, feline immunodeficiency virus, equine infectious anemia virus, bovine leukemia virus, caprine arthritis virus; Corona viruses (canine coronavirus, feline enteric coronavirus, feline infectious peritonitis virus; Babesia spp (B. canis, B. gibsoni); Dirofilaria spp (heartworm); other parasitic diseases (fleas, ticks, roundworms, tapeworms, hookworms, Strongyles and other intestinal parasites); and Chlamydia antigen (PCR testing).
2) International: Same as above plus Leishmania spp; Trypanosoma spp.; Anaplasma spp; Yersina pestis.

10. Test 10: Other Diagnostic Tests

Patient phenotypic descriptors and genotypic descriptors/background, plus any or all selected tests from the following list:

Pathology (anatomic, histological, cytologic, immunohistochemical, electromicroscopy, FACS); blood typing; bone marrow analysis and specific immunohistochemical staining; RFLP and PCR testing (applicable to many of the above categories); IFA and FA testing; ELISA testing, cell cytotoxicity testing, cytokine testing (see Test 3, other cytotoxic cell and mitochondrial tests); markers of neoplastic and paraneoplastic change (cancer); neurotransmitters including serotonin, gamma-aminobutyric acid (GABA), glutamate, dopamine, glycine, aspartate, acetylcholine, norepinephrine, histamine, substance P, vasopressin, vasoactive intestinal peptide, neurotensin, or other neuropeptides; and amino acid profiling.

D. Comprehensive and Cumulative Database for Animal Health

Comprehensive and cumulative data profiling is utilized over time to allow one to predict the specific nutritional management interventions that can assist in the care and management of the very earliest stages of specific abnormalities or trends that have been identified in the health profile of animals, thereby extending and improving their health and longevity. This is an unique approach to scientifically and medically determine by comprehensive and cumulative laboratory and/or genetic profiling of individual animals and animals within specified defined groups to permit intervention in prevention, management and treatment of general and veterinary medical health care.

Specifically, the present invention directs the outcome of the laboratory and/or genetic profiling to nutritional and nutritional supplement management of the specific identified abnormalities and trends over time. This is not only important but also practical because nutritional intervention and management is relatively inexpensive, non-invasive and easily accepted by the pet owner and the veterinary professional making these recommendations. Thus, the present invention involves a dynamic method and system of managing the health care, well-being and nutritional requirements of companion animals.

The nutritional regimen is at least related to the nutrient or caloric composition needed for the subject, or the food allergies and food intolerances of the subject. Nutritional regimen can also be therapeutic and/or preventative intervention of the disease and/or disorder. The nutritional regimen may include pre-selected, pre-prepared food, treats, drinks and or nutritional supplements, i.e., nutraceuticals, anti-oxidants, vitamins or minerals. Yet further, nutritional regimens may also include holistic treatments or exercise.

For simplicity, an example is described for a dog, however, the present invention can be equally applicable to a cat or any other companion animal. To begin, a database relating to the dog species generally, and a database relating to a selected group, for instance, the breed, of the dog is used. Data is obtained relating to the particular dog subject, and this data includes diagnostic laboratory test data, and ideally comprehensive and selected diagnostic laboratory data relating to the dog. The database of the selected dog group and the breed, for example, is related to the data of a particular subject dog by a computer. For example, the database of the selected group of the species is at least one of breed, age, sex, size, weight, performance use, or geographical location. There is then determined, based on this relationship, a regimen for the management and health care of the dog subject.

Diagnostic laboratory test data is a comprehensive general health profile and at least one selected diagnostic profile for a selected subject animal. The laboratory data for the subject is ideally obtained over time from the same laboratory. This is likely to enhance the uniformity of the data, and render the determinations more accurate and predictive of health, nutritional requirements, temperament, and longevity.

The database of at least one of the species or the group is periodically updated thereby to obtain cumulative data of the dog species or group within the dog species. Both of these databases generally are be used, and both are updated to obtain the cumulative data. In some cases, only one of the databases is used and/or one of them is periodically updated.

The data of the dog subject is also periodically updated. Overall there is obtained cumulative data of the dog subject, species or group. The updating picks up data drift or data trends within different populations of the particular dog subject, the groups (for instance, breed) and the species (for instance, the dog generally as a species) over time. This allows for the review and oversight of the database so as to be substantially or essentially current.

The data of the dog subject is compared to substantially or essentially current data. Similarly, by retaining a historical record of the dog subject's data and relating this to the updated databases, the accuracy with which the management of the health care and wellbeing, and the development and design of nutritional requirements or therapeutic and maintenance interventions is significantly enhanced. In this manner, for instance the food, supplements, nutraceuticals and the like, can be modified by additions and/or subtractions of components based on the determined relationship, since these cumulative and dynamic databases and data analytes change over time, whereby the determined relationship is significantly enhanced. Management of the dog subject in one or all of these respects is dealt with a high level of precision and predictability.

The computer is at least one of an expert system or interrelationship program or network for determining data base and data relationships. This can be a system such as a neural network, or other statistical sampling systems and networks, and is discussed in more detail in later sections of this application.

The determination of the health care, well-being, nutritional or other therapeutic requirements and suggestions for promoting and maintaining health of the dog is reported on a communications network including the Internet. There is a payment procedure for the report which is achieved through the Internet.

The system also permits for the access to the genetic and/or phenotype data through a password and a system whereby access to the data generates a fee. This system also provides for a situation wherein payments can be made by credit card for requests to perform health assessment profiles and secure genomic mapping and genetic screening information. Such bioinformatics system can also permit for the automatic payment for such services and products to the banking system of the database or laboratory. As such, the database may require that the payments be guaranteed, for instance by supplying a credit card number with a request for performance of services and a product, and for the retrieval of such data.

A user can submit a request to the database in any number of ways. For example, the request can be submitted via on-line direct connection, namely through a computer network such as the Internet. An intermediate researcher such as a veterinarian or scientist other than the owner could also submit the request on behalf of the owner using the e-mail capabilities of the central database system. Alternatively, the user can submit the data via an interactive voice response unit coupled to the database system of the supplier. In some situations, the database supplier can decide whether to supply the health assessment information and/or genomic mapping and genetic screening information based on the criteria of the user or its intermediary agent. Such user or intermediary agent can be notified of the decision via the interactive response unit or a live operator.

The user or agent can log into the database system and obtain the necessary records relating to an animal physical health and/or genetic ancestry or offspring. The database system can transmit in real time or on a periodic basis as determined, thereby, providing information regarding the health assessment or the genetic background and forward this information to the user and/or its intermediary agent.

The data storage devices of the invention include a variety of databases including a database relating to the phenotypic data of a particular species, a database relating to health assessment or other phenotypic data of particular animals in a particular species, and genetic characteristics of different species and different family trees relating to different species. The family trees contain information including the origin, genomic map, and parental lines of a species and records of health and performance of a species. These databases are interrelated in an analytical manner and in accordance with different algorithms of permutations and probabilities to facilitate useful output information based on the combination of data in the genotypic and the phenotypic databases, and the selected databases.

In further embodiments, it is envisioned that the method is not a dynamic method. For example, the method may involve a single DNA test in which the result will not change with time and/or treatment. One such single test includes, but is not limited to a DNA test, which indicates whether or not the animal has the genetic predictors of the disease, i.e., the presence and/or absence of any DNA marker associated with the disease. The data from the DNA test can then be interpreted in view of the database to determine the nutritional regimen. Thus, the present invention may also involve a sequential method and system of managing the health care, well-being and nutritional requirements of companion animals.

It is also contemplated that this sequential method may not require the assistance of a database to interpret the data and/or determine the nutritional regimen. The single test provides a positive or negative result, which indicates the presence and/or absence of a disease and/or disorder. Based upon the data from the single test, one can determine the nutritional regimen without a database.

E. Overall System

Below is a description of the system that is used in the desired methods to manage the health care, well being and nutritional requirements of the companion animal.

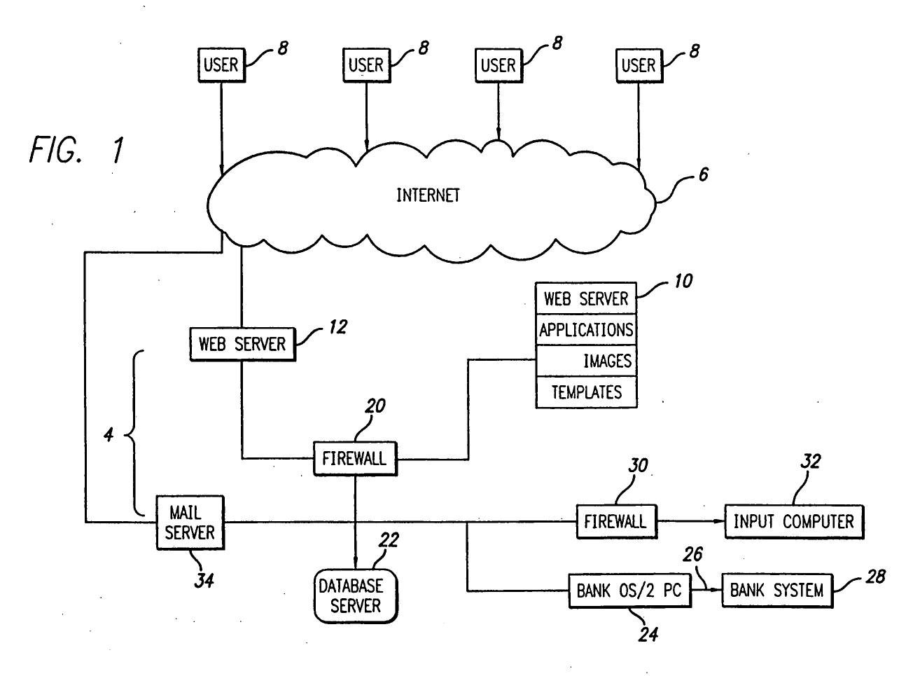

FIG. 1 is an overview of the web-based system to provide access to the invented database management system. With this system multiple users, for instance, remote users 8, access the web site 4 using the Internet 6. Each of the users 8 has a computer terminal with the appropriate software for accessing Internet. The users 8 may be unknown to the web server computers 10 and 12. Each user 8 is allowed to browse the web site and explore how the system functions.

There are several aspects to maintain security of information maintained in the database server 22 and a banking system 28. A firewall 20 prevents any user 8 from accessing any of the components behind the firewall 20. In this way the users 8 have access to the web server computers 10 and 12, but only have access to the database server 22 through the firewall 20. The database server 22 maintains, among other things, various database fields with respect to each of the health profiles of subjects and the genetic information of a subject and groups. The database 22 maintains the services with a designation associated to determine what health assessment data and genetic data can be browsed by the users 8. Each of the web server computers 10 and 12 allow users 8 to view subject and group categories and actual services and data products which are available from the database.

The web server computers 10 and 12 can be identical and can be duplicated as additional load or growth on the system occurs. The web server computers 10 and 12 share the responsibility for servicing the users of the site. This arrangement provides for expandability of the system by merely adding additional web server computers as necessary.

Preferably, the system includes an appropriate computer terminal 24 for interfacing with independent financial institutions which are connected on-line via the serial connection 26 to the financial institution computers 28. This allows automatic real time confirmation of the access of health profile and genetic data services and products. Once a user requires access to a product or service, the user goes through an identification or registration process and the exchange of financial information to allow for credit or debit card payment of the purchase. This is verified, confirmed and authorized by the appropriate bank system institution 28. Confirmation of the purchase or deposit of data, or a service is made by a mail server 34 which sends an E-mail to the user 8 confirming the purchase or deposit. The mail server 34 allows for mail to be received and sent out. Security of the various databases is maintained. Alert messages are generated when an unauthorized access is attempted. Verification messages, authorization messages and confirmation messages are generated as appropriate.

The database server 22 is also designed to interact with an input computer 32 operated by a CDPR. A firewall 30 serves to prevent unauthorized access to the database server 22 or to the input computer 32. The input computer 32 can input health profile data and genetic data to the database, after appropriate access and/or passwords are entered into the system. Similarly, users 8 through their own computers can use appropriate access codes and passwords to access input data to the database server 22. This is tightly controlled for security reasons. The data may only be added to an independent sub-database of the data server 22, and only after scrutiny by the CDPR operator of the database through input computer 32, will this data from users 8 be subsequently added to the main database server 22.

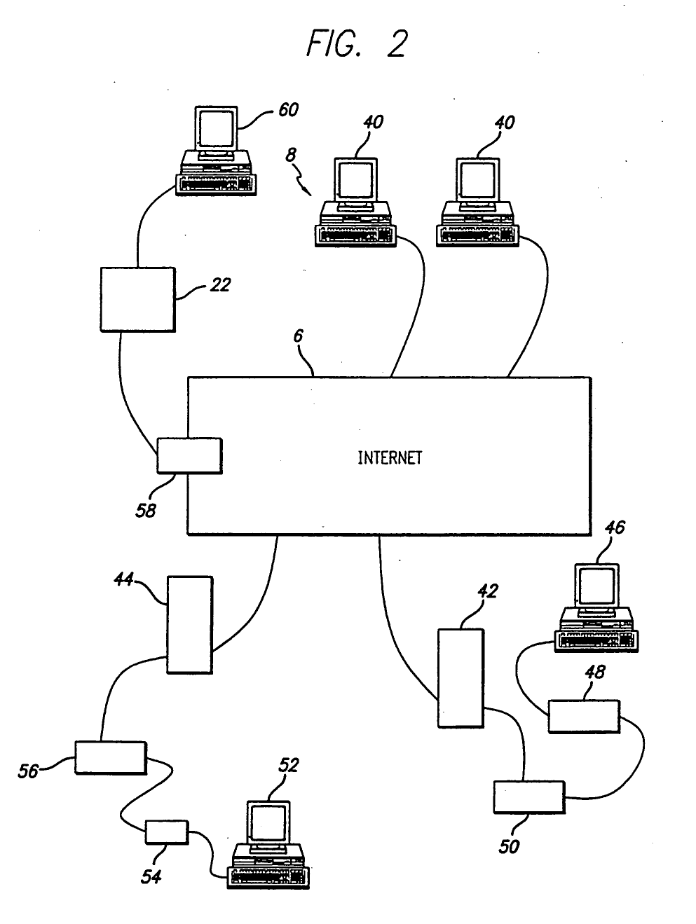

FIG. 2 is an illustration of the Internet and its use in the system of the invention. The Internet 6 is a network of millions of interconnected computers 40 including systems owned by Internet providers 42 and information systems 44 such as America Online™. Individual or corporate users may establish connections to the Internet in several ways. A user on a home PC 46 may purchase an account through the Internet provider 42. Using a modem 48, the PC user can dial up the Internet provider to connect to a high speed modem 50 which, in turn, provides a full service connection to the Internet. A user 52 may also make a somewhat limited connection to the Internet through a system 20 that provides an Internet gateway connection 54 and 56 to its customers. The database 22 is also connected into the Internet 6 through an appropriate modem or high speed or direct interface 58. The database 22 is operable and maintained by the CDPR operator computer 60. Users of the databases of the invention would access the Internet in an appropriately selected manner.

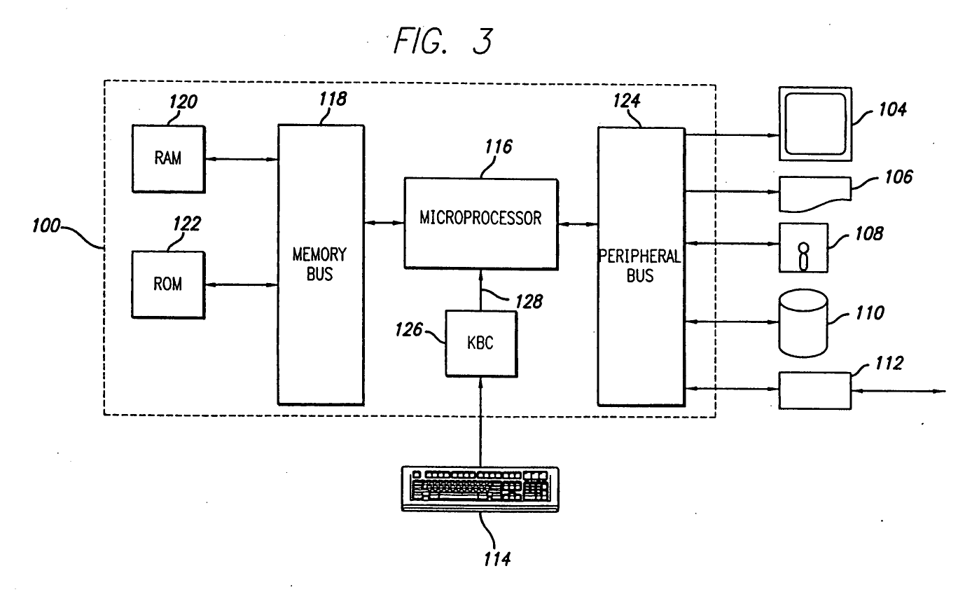

FIG. 3 is a block diagram of an exemplary computer system 100 for practicing various aspects of the invention. The computer system 100 includes a display screen or monitor 104, a printer 106, a disk drive 108, a hard disk drive 110, a network interface 112, and a keyboard 114. The computer system 100 includes a microprocessor 116, a memory bus 118, random access memory (RAM) 129, read only memory (ROM) 122, a peripheral bus 124, and a keyboard controller 126. The computer system 100 can be a personal computer, such as an Apple computer, i.e., an Apple Macintosh™, an IBM™ personal computer, or a compatible, a workstation computer, such as a Sun Microsystems™ or Hewlett-Packard™ workstation, or some other type of computer.

Microprocessor 116 is a general purpose digital processor which controls the operation of computer system 100. Microprocessor 116 can be a single-chip processor or can be implemented with multiple components. Using instructions retrieve from memory, the microprocessor 116 controls the reception and manipulation of input data and the output and display of data on output devices.

Memory bus 188 is used by the microprocessor 116 to access RAM 120 and ROM 122. RAM 129 is used by microprocessor 116 as a general storage area and as scratch-pad memory, and can also be used to store input data and processed data. ROM 122 can be used to store instructions or program code followed by microprocessor 116 as well as other data.

Peripheral bus 124 is used to access the input, output, and storage devices used by computer system 10. These devices include the display screen 104, printer device 106, disk drive 108, hard disk drive 110, and network interface 112. The keyboard controller 126 is used to receive input from the keyboard 114 and send decoded symbols for each pressed key to microprocessor 116 over bus 128.

The display screen or monitor 104 is an output device that displays images of data provided by microprocessor 116 via peripheral bus 124 or provided by other components in computer system 100. The printer device 106 when operating as a printer provides an image on a sheet of paper or a similar surface. Other output devices such as a plotter, typesetter, etc. can be used in place of, or in addition to the printer device 106.

The disk drive 108 and hard disk drive 110 can be used to store various types of data. The disk drive 108 facilitates transporting such data to other computer systems, and hard disk drive 110 permits fast access to large amounts of stored data.

Microprocessor 116 together with an operating system operate to execute computer code and produce and use data. The computer code and data may reside on RAM 120, ROM 122, or hard disk drive 120. The computer code and data could also reside on a removable program medium and loaded or installed onto computer system 100 when needed. Removable program mediums include, for example, CD-ROM, PC-CARD, floppy disk and magnetic tape.

The network interface circuit 112 is used to send and receive data over a network connected to other computer systems. An interface card or similar device and appropriate software implemented by microprocessor 116 can be used to connect computer system 100 to an existing network and transfer data according to standard protocols. As such he computer system is connectable through an interface device with the Internet 6.

Keyboard 114 is used by a user to input commands and other instructions to computer system 100. Other types of user input devices can also be used in conjunction with the present invention. For example, pointing devices such as a computer mouse, a track ball, a stylus, or a tablet can be used to manipulate a pointer on a screen of a general-purpose computer.

The present invention in relation to the animal database management of data can also be embodied as computer readable code on a computer readable medium. The computer readable medium is any data storage device that can store data which can be thereafter read by a computer system. Examples of the computer readable medium include read-only memory, random-access memory, magnetic data storage devices such as diskettes, and optical data storage devices such as CD-ROMs. The computer readable medium can also be distributed over network coupled computer systems so that the computer readable code is stored and executed in a distributed fashion.

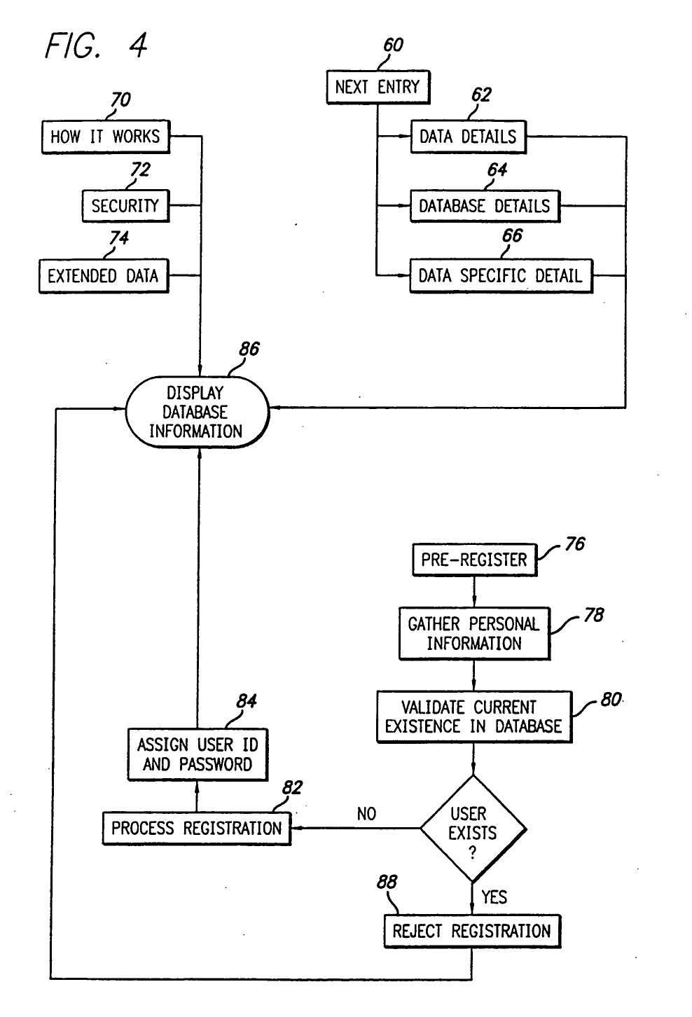

FIG. 4 illustrates a browser system for use with the database system of the invention. A browser goes through a number of preliminary screens and logic steps, and reaches a screen 60 entitled “Next Entry”. This screen provides data details or information generally indicated as 62. Clicking on any of these categories allows the user to review database details 64, data specific details as generally indicated by 66. In this way, the user can index through a number of screens to get information regarding the different databases of the system. In addition, clicking on any of the triggers 70, 72, 74 and 76 is possible. These correspond to HOW IT WORKS, SECURITY, EXTENDED DATA and PRE-REGISTRATION. Clicking on trigger 70 provides the user with information on how the process works, explains the system, and provides details on how the user can participate in the database and obtain data or input data. Clicking on trigger 72 provides details regarding security of the system and automatic payment. In some cases, products and services are offered with extended data and clicking on trigger 74 which can provide details of the extended data and explains that this may only be available on certain services or products.

Trigger 76 allows a user to pre-register and obtain user ID number. This ID number is combined with financial information retained in the database in an encrypted form. The pre-registration trigger 76 follows with step 78 which is to gather personal information such as credit card number and expiry date to allow for automatic payment. Step 80 is to validate a current existence in the database, if this occurs. With a negative answer, the user is directed into a registration process indicate as 82. A user ID is assigned and a password is entered. This information is maintained in a portion of the database 22. At 84 the user is provided a screen identifying the user ID at screen 86. If the user already exists, the registration process is rejected at 88 and the user is advised of the information at the display 86. The screen at 86 would also represent the information which is available in the database 22.

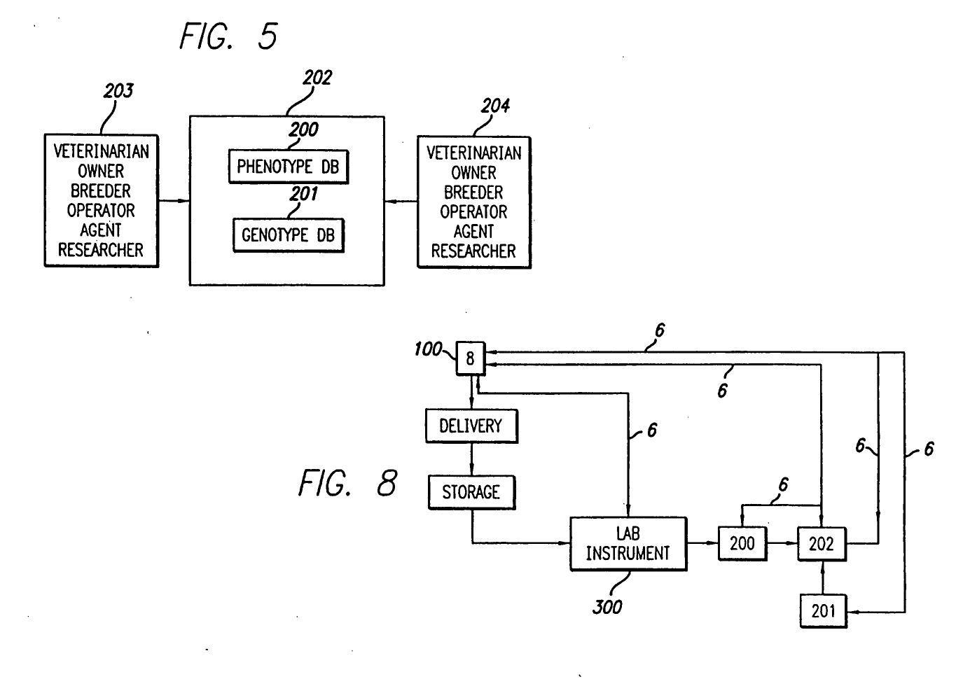

In FIG. 5 there is shown a basic block diagram of the components making up the CDPR. There is the phenotype database or physical health database 200 and a genotype database or genetic information database 201. These are contained in part of the overall CDPR database 202. User input 203 can be obtained from a remote user such as a veterinarian, owner, breeder, or the operator of the database, an agent or researcher. The output from the database 204 could be to the veterinarian, owner, breeder, operator, agent or researcher.

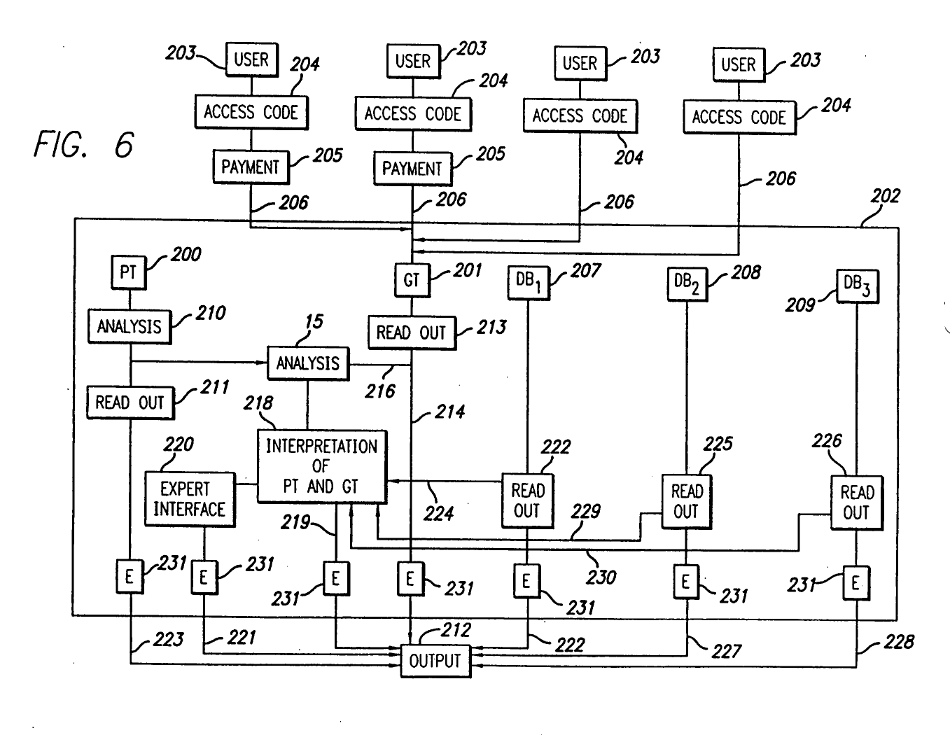

FIG. 6 shows a relationship for retrieving data from the database 202. The user 8 is represented here as a veterinarian, owner, breeder, operator, or researcher 203 who accesses the CDPR 202 accesses a first screen through a computer network 6 which inquires about information about the user. An access request message is sent, and an appropriate access enabling message is transmitted. The user 203 can obtain partial or full access to the CDPR 202 according to the scale of authority given to the user 203 to access data. There is a computer program system 205 to ensure that payment is made as appropriate before access to the CDPR 202 is granted. In some situations, the appropriate access code 204 can permit bypassing the payment requirement 205 as indicated by line 206. Payments 205 through the computer program can be effected by a credit card entry and automatic transfer to a financial institution on behalf of the operator of the CDPR 202. Such payment for access to the database is effected by a system which is well known in the art. The financial institution will appropriately credit the operator of the CDPR 202 in a financial manner as established between the operator and the financial institution.

Within the CDPR 201 there is the ability to access the physical health phenotype database 200, the genotype database 201, and other databases 207, 208 and 209, respectively. The phenotypic and genotypic information together with other database information can be presented on a single screen or monitor or other viewing means, for instance, hard copy format. The access therefore can be to multiple databases contained within the CDPR 202. After accessing the physical health database 200, the user obtains an analysis report from module 210. The user is then able to read the analysis as indicated by 211 and output the analysis from the read-out 211 as indicated by output 212. The output 212 can be a computer screen read-out, fax or voice information.

The physical health or phenotype database 200 is subject or group specific. In other words, the data obtained in that database is specific to a particular animal or animal group (breed, family, species, etc.) which has been the subject of a laboratory or research biological examination such that fluid or tissue samples have been subject to analysis in one or more laboratory or research environments. These biological reports can include those from specimens of blood, urine, other body fluids, skin, eyes, skeletal and other tissues. The PT database 200 has the ability to store the subject specific information as required within the CDPR 202.

The genotype specific or genetic disorder or disease data is retained in the database 201 within the CDPR database 202. This data is either subject specific, family specific, breed specific, species specific, disorder specific, or disease specific, and is group or subject specific. The user can access the genotype database 201 and obtain a read-out 213 which can then be transmitted along line 214 to an output 212 in the same manner that the physical health assessment is obtained as an output.

In an alternative approach, the reader can request an analysis 215 from the genotype database as indicated by line 216. This analysis can receive data along line 217 from the analysis information of the physical health assessment. Interpretation of the PT and GT can be obtained as indicated by 218, and this can then be outputted as indicated along line 219. The interpretation of PT and GT 218 can be performed by an algorithm relating to the coefficients and predictability of information relating to disorders, disease and longevity when considering the data from the two databases PT 200 and GT 201. This can be done automatically and outputted along line 219, or there can be an expert interface 220 using skilled personnel to interpret the data of block 218, and this can, in turn, be outputted along line 221 to the output 212.

Database 207 can be a genetic marker database, and the information from that database can be directly input into the output through a read-out 222 and 223 to the output 212. Alternatively, the data from database 207 can be added to the interpretation section 218 of the physical health and genetic information by directing the data along line 224. This data can then be made the subject of the output along the line 219 and 221 as required.

Similarly other databases 208, 209, respectively, have read-outs 225 and 226 which can be directly coupled along lines 227 and 228 to the output, or can be directed optionally along lines 229 and 230 to the interpretation module 218. It can then be the subject of interpretation for an expert interface 220 review which is, in turn, made the subject of the output 219 and 221.

In each of the output lines 219, 221, 222, 223, 227, 228, and 214 there is also provided an encryption program 231 which can be optionally used in the system. The output 212 can include paper, electronic, or voice read-out as is required.

In this manner, the output 212 provides a compilation which combines the physical health and genetic information relating to a subject, the breed, disease, disorder and lifespan, thereby enabling the receiver of the output 212 to use the compiled information in a manner to facilitate breeding criteria which can be important in relation to animals which are usually inbred or line bred. The information can also be used to facilitate on-going monitoring of particular subject animals. The data from this system can be used to manipulate and regulate breeding, health, and longevity effectively among animals.

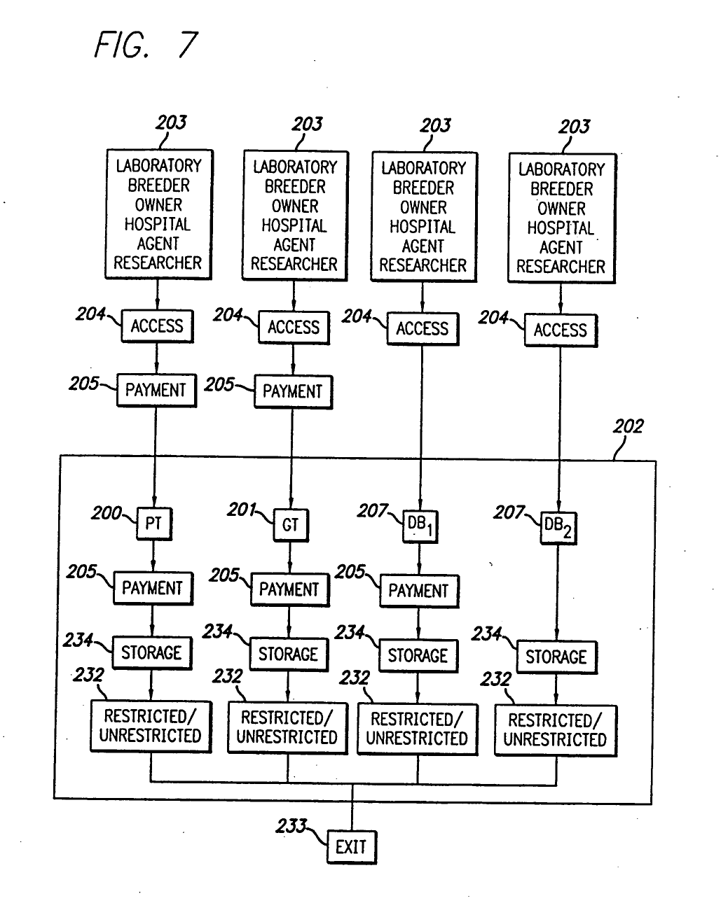

The system of the invention is further described with regard to FIG. 7, which is a system for inputting data to the CDPR 202. Here multiple users 203, which can be a remote user such as a laboratory, a breeder, an owner, hospital, agent, or an operator of the CDPR 202 accesses the system through module 204 which, in turn, accesses the CDPR 202. Appropriate access request and access enable messages are sent. Within the CDPR 202 there is a physical health or phenotype module 200, a genetic or genotype data module 201, and other database modules 207, etc. After accessing the CDPR 202, additional data can be added to the modules 200, 201, 207, etc. through any of the users 203, if authorized. Depositing data into each of the modules 200, 201 and 207 can optionally require the payment to the operator of the CDPR 202 as is indicated by block 205. This system can function in the same manner as the retrieval of data from CDPR 202.

The stored data in each of the blocks 200, 201, and 207 can be set up as indicated by block 232 in a manner which is restricted or unrestricted to selected users 203. This may be necessary according to the protocols governing the inputted data to the different databases. In some cases, the waiving of deposit fees is made in the interest of freedom of the database to subsequent users who wish to retrieve data from the database. After storage of the data as indicated by block 234, the user 203 exits CDPR 202 as indicated by block 233.

As is apparent, the physical health or phenotype profile of subject animals is dynamic and grows as more data is added into the system. Likewise, the genetic or genotype database also grows as increasing research of particular subjects, breeds, and the like is obtained. The deposit of new information into the CDPR 202 is regulated in a manner that the data cannot distort the databases 202 in an in appropriate manner. Likewise, users 203 cannot access the secured databases within CDPR 202 in an inappropriate manner.

Different algorithms regulate the relationship between the health profile, the genetic data, and other data relating to animals. These algorithms determine the probabilities, possibilities, and likelihood of disorders and disease in subject animals and offspring animals. They are used as predictors of the future evolvement of health of the animal.

In one example the genetic influence on behavior and behavioral disorders accounts for less than half of the phenotypic expression of behavior and behavioral differences. However, behavior is the most complex phenotype, because it reflects not only the functioning of the whole being but also is dynamic and changes in response to environmental influences. These results are most dramatically seen in purebred animals because they have been inbred and line-bred to select for a particular behavior and conformation, even though the genotype of purebred breeds shows almost no variation over 100 years. Examples of this are all the different purebred dog breeds which currently exist, and have widely disparate size, weight, temperament and lifespans.

Accordingly, if the results of a mostly phenotypic database indicate abnormal thyroid function, then by relating this to the mostly genotypic and combined database categories of breed, age and sex, it is possible to determine whether the subject has or does not have heritable thyroid disease, or is likely to develop this condition within a predicted period of time.

Similarly, if the phenotypic database indicates elevated blood and urine glucose levels, then by relating this to the genotypic and combined database categories of weight, age, sex, breed and reproductive history, it is possible to determine that the subject has diabetes that is likely to be of an heritable basis.

Another example relates the phenotypic database indicating low blood von Willebrand factor level to the genotypic and combined database categories of breed, age, sex, and clinical and family history, whereby it is possible to determine whether the subject has the inherited or acquired form of von Willebrand disease.

Analyzing the data from the CDPR 102 in the manner of the present invention permits for genetic screening, health assessment profiling, and the diagnostic, prophylactic, and therapeutic management of animals.

F. Inter-Relationship of the Phenotype and Genotype Database

The determination of the interrelationships between individuals or groups of individuals in the database can use any one of a number of computerized or other methods of analysis, simple or complex, including such things as neural networking or other kinds of relational technology evaluative databases.

An exemplary server performs all the operations of a conventional database system and performs additional operations in accordance with the present invention as has been discussed, in the previous section, which is incorporated herein. The server includes a central processing unit (CPU) together with associated memory for processing information about different animals species and history. The inquiries concern animals species and history and inquiries and requests for health profiling and genetic information, and providing health profiles and genetic information. The CPU is coupled to the database and to users via a communications port. The CPU is also coupled to an electronic mail processor for processing and storing (in a storage device) e mail messages transmitted between the CPU and various agents, users and the like. The CPU is further coupled to a data storage device. A data storage device may include a variety of the databases. The system permits for the requesting, storing and providing of data with respect to animal phenotypic information and genetic information. The format and content of the databases have been discussed in detail above and is incorporated herein.

FIG. 8 presents an overview of the laboratory instruments apparatus, system, and method operable with the present invention in relation to a CDPR 202. The present invention allows access by remote users with computers or processors 100 to receive and access data on specimens. Using the Internet 6 or other computer network or communication link capability, the remote user 8 sends a message to request access to the services provided by the laboratory or operator which has a CDPR 202. If access to the CDPR 202 is granted, a message is sent to the remote user computers 100. This message includes instructions enabling the remote user 8 to define and access data stored in the CDPR 202.

In one form of the invention, the desired data is based on the submission of test specimens of a specific animal to the laboratory. In some other cases health profile test data 200 can be inputted into the CDPR 202 having the genetic database 201. The CDPR 202 can perform an analysis and correlation between the health profile database 200 and the genetic database 201.

Using the communications link, the remote user 8 communicates with the laboratory or the CDPR 202. Specimens can be packaged and physically transported to the laboratory site via commercially available common carriers, such as the postal service or courier services. When the packages arrive, the laboratory places them in storage, or the tests are performed. Instruments 300 perform the tests to obtain data as specified by the remote user 8. The biohazardous samples can be disposed of a waste material. The test results, or output is provided as part of a health profile database 200 of the CDPR 202 and is available to the remote user 8.

If desired, the remote user 8 can arrange to have the data stored in the CDPR 202, made available to other remote users 8. The remote user 8 can also request the laboratory to perform analysis on the health profile data 200 generated.

In one embodiment, the communications link is a computer network and the message transfer modality is, for instance, the Internet 6, and/or an Intranet and/or an Extranet. The network systems are particularly suited to the application described herein since it offers global or widespread accessibility and high speed data transfer of large amounts of information.

A security unit allows remote users to designate who has permission to view or use their data. Feasible options for these information management requirements include: access by the submitting remote users only, access by certain designated researchers and collaborators, time-embargoed data followed by wider access, and unrestricted access by all. A commerce unit can implement functions related to the business aspects of the CDPR facility, including billing, inventory management of support materials.

A multimedia unit comprises means to store, manipulate, and present audio, graphical, video information. This information may include a video explaining how the CDPR is used, a visual depiction of the data, methodology, or a comment regarding the background of the data. The multimedia unit may also implement subscription functions, so that updated data automatically provided to remote users or other interested parties.

The operations performed by the present invention begins when the controller receives an access request message from the remote user via a communication link. Using information in the access request message and any other available information, the controller determines if the remote user is authorized to access the CDPR 202. If so, an access enabling message is transmitted from the controller to the remote user 8. The access enabling message can comprise a set of computer instructions transmitted over the Internet 6 which is downloaded into the remote user memory for execution by the remote user processor. These instructions may be enabling, that is, they may allow direct communication between the remote user 8 and the CDPR 202 with no further need for the controller. In another embodiment, the access enabling message may simply comprise a password or other enabling message which allows the remote user 8 to proceed. The remote user 8 can access or submit data to the CDPR 202 according to different protocols and regimes and security arrangements.

Different forms of expert system computing and software programming can be used to determine the relationship of the data bases and data. Parallel distributed processing, and neuromorphic systems, such as neural networks can be used. They are good pattern recognition engines and robust classifiers, with the ability to generalize in making decisions about imprecise input data. There are multitudes of different types of networks such as a multilayer perception which is generally trained with the backpropagation of error algorithm, learning vector quantization, radial basis function, Hopfield, and Kohonen. Some are feedforward while others are recurrent (i.e., implement feedback) depending on how data is processed through the network. Some may require training while others are unsupervised or self-organizing. This can be implemented in software or in specialized hardware.

Alternatively or additionally fuzzy logic can be used due to the dynamic nature of the data applications, rules and functions. Such logic is adaptive to the changing environment. This logic and the neural networks can be integrated in the system.

Adaptive Logic Networks technology is an effective alternative or additional technology. The Adaptive Logic Network is neurocomputing capable of modeling complex non-linear systems by using piece-wise linear data The inputs to an Adaptive Logic Network may be the data from large databases as described, observations recorded by a scientist, veterinarian or owner. The outputs of an Adaptive Logic Network can be used for analysis, prediction, or real-time management.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Temperament and Longevity

Test panels Nos. 1, 2, 3, 8 and 10 set out in the above section can be used to obtain data for this Example.

Briefly, characteristics related to the temperament of the animal, which impacts on its longevity, are determined. Biological laboratory test data from a bodily fluid or tissue of an animal are analyzed. Such test data relate to the level of neurotransmitter activity of the animal. The data relate to at least one of the value of serotonin, gamma-aminobutyric acid (GABA), glutamate, dopamine, glycine, aspartate, acetylcholine, norepinephrine, histamine, substance P, vasopressin, vasoactive intestinal peptide, neurotensin, or the other neuropeptides of the animal. The value should fall within predetermined levels as a predictive determinant of the animal's temperament (passivity, assertiveness, or aggressivity).

Methods for measuring neurotransmitters are well known in the art. Neurotransmitters such as serotonin, epinephrine, norepinephrine, glutamate, and GABA can be measured by standard immunochemical techniques involving commercially available antibodies, either polyclonal or monoclonal. Such antibodies are commercially available from sources such as Sigma Chemical Company (St. Louis, Mo.). These immunochemical techniques can involve either radioimmunoassay or other well-established assay techniques, such as ELISA (enzyme-linked immunosorbent assay). These neurotransmitters can also be measured by standard non-immunochemical techniques such as gas chromatography. Neuropeptide neurotransmitters are preferably measured by immunochemical techniques.

### Example 2

### Immune Stimulation and Cellular Inflammatory Response

Test panels Nos. 1, 3, 4, 8, 9 and 10 set out above can be used to obtain data for this Example.

Characteristics related to at least one of the immune stimulation reaction, evidence of neoplastic or paraneoplastic change, or the cellular inflammatory response of the animal are determined. Biological laboratory test data from a bodily fluid or tissue of an animal are analyzed. The test data relates to at least one of cell cytotoxicity markers, cytokine and chemokine levels, immunoglobulin levels, type and amount of lymphocyte subsets and lymphocyte markers, and markers of neoplastic or paraneoplastic change of the animal. The value should fall within predetermined levels as a determinant of the immune stimulation reaction, neoplastic or paraneoplastic change, or the cellular inflammatory response.

Methods for measuring lymphokines and other cytokines are well known in the art. These compounds are typically measured by immunochemical techniques using commercially available monoclonal antibodies or other methods.

### Example 3

### Inherited Organ Dysfunction or Dysplasia

Test panels Nos. 1, 3, 5, 9 and set out in the above section can be used to obtain data for this Example.

Characteristics related to inherited organ dysfunction or dysplasia of the animal, at least one of which is neuronal, neuromuscular or renal are determined. Biological laboratory test data from a bodily fluid or tissue of an animal are analyzed. The test data relate to an amino acid, carbohydrate, lipid or other metabolic component, body fluid or tissue marker of the animal. The data includes obtaining data related to at least one of the value of the methyl malonic acid, the fucose-containing cell metabolites, blood or urine urate or uric acid metabolites, normoglycemic glycosuria, mannosidase containing cell metabolites, amino acid uria, amyloid deposition in tissues, neuronal ceroid lipofuscin deposition, and deposition of gangliosides and other lysomal storage substrates of the animal. The value should fall within predetermined levels as a determinant of the inherited organ dysfunction or dysplasia.

### Example 4

### Autoimmune Thyroiditis

Test panels Nos. 1, 2, 3 and 10 set out in the above section can be used to obtain data for this Example.

Characteristics related to autoimmune thyroiditis of the animal are determined. Biological laboratory test data from a bodily fluid or tissue of an animal are analyzed. The test data relate to a genetic marker for automimmune thyroiditis of the animal. The data relates to at least one of the results of a comprehensive thyroid antibody test profile, DNA fingerprint (the gene map), and markers for immunoglobulin receptors on B-cells, T-cell receptors, and protein products of the major histocompatibility complex (MHC) genes (Class I and II allellic HLA, DLA or equivalent antigenic specificities of the animal. Test assays to screen for MHC genes include restriction fragment length polymorphism (RFLP), polymerase chain reaction (PCR) RFLP, PCR sequence-specific oligonucleotides (SSO) and PCR sequence-specific primers (SSP). The value(s) should fall within predetermined levels as a determinant of autoimmune thyroiditis.

### Example 5

### Mammary Cancer

Test panels Nos. 1, 2, 3 and 10 set out in the above section can be used to obtain data for this Example.

Characteristics related to presence of or susceptibility to mammary cancer of the animal are determined. Biological laboratory test data from a bodily fluid or tissue of an animal are analyzed. The test data relate to estrogen (estradiol-17β), estrogen receptors, interleukin (IL) 6, progesterone, and progesterone receptors. The value should fall within predetermined levels as a determinant of the presence of or susceptibility to mammary cancer.

### Example 6

### Immune Surveillance

Test panels Nos. 1, 3, 5, 6, 8, 9 and 10 set out in the above section can be used to obtain data for this Example 6.

Characteristics related to the tissue environment of the eye and brain (ocular and blood-brain barrier) which are sites protected from the normal immunologic surveillance mechanisms are determined. Biological laboratory test data from a bodily fluid or tissue of an animal are analyzed. The test data relate to the soluble and cellular immune inflammatory response mediators (cytokine and chemokine levels, immunoglobulin levels, and lymphocyte susbset markers). The value should fall within predetermined levels as a determinant of integrity of protected immune surveillance mechanisms.

### Example 7

### Inherited Bleeding Disorders

Test panels Nos. 1, 7, and 9 set out in the above section can be used to obtain data for this Example 7.

Characteristics related to the tendency to bleed excessively are determined. Biological laboratory test data from a bodily fluid or tissue of an animal are analyzed. The test data relate to a comprehensive assessment of the hemostatic and coagulation function. The value should fall within predetermined levels as a determinant of the presence of bleeding disorder.

### Example 8

### Comprehensive and Cumulative Database for Animal Health

An initial database, from a recent temporal period made on a group of healthy dogs, may use physical characteristics, health history, and comprehensive laboratory data of these dogs for a specific geographic area such as the U.S.A. and Canada, but also other geographic areas could be used. Additional databases are developed for other countries. Generally the same laboratory is used to generate the database. The temporal retrospective database are augmented in an on-going fashion with prospective data that continues to accumulate over the future testing years. Results are analyzed from the temporal retrospective database and then are periodically reanalyzed every 6-12 months depending on the size of the database to search for any trends or drift in the values of specific analytes over time.

This is an important database to accumulate because the presence of drift over time means that subsequent studies of the same or other animals, whether they be healthy or have diseases, disorders or changes in lifestyle, diet or other parameters including reproduction, or performance use need to take such drift in the database into account in order to accurately interpret the values obtained. Animals of specific breed or type characteristics, size, age, weight, performance level, lifestyle, geographic location have their laboratory profiles and physical characteristics and health history entered into a database that starts from entry into the system and continues on a regular basis over time, preferably at least annually. Also entered into this database are puppies that are tested for the first time at about six months of age, pre-puberty, and then before puberty, preferably in anestrus females, and then annually thereafter in a comprehensive manner to establish a cumulative laboratory database for the individual animal.

These data are put into a group-specific database for the breed characteristics or the activity characteristics or any other parameter that is useful to group together for analytical purposes. This method of gathering comprehensive and cumulative data permits not only analysis of individual animals, whether they be healthy or expressing some stage of disease or disorder, but also allow analysis of their membership in a group. When the group is analyzed it provides a database for predictive laboratory value expectation for similar members of the group. By developing these databases in a cumulative manner the trends for particular analytes or groups of analytes predictive of organ function, for example, can then be compared within individual animals, healthy or diseased, with that of the retrospective and prospective healthy animal database to look for differences in trends. Those differences in trends, as well as differences in individuals or groups of animals, can then be used as a predictor of health, disease and longevity.

Once trends or changes are identified within individual animals, or within the related groups of animals, or within specific analytes or groups of analytes from a database, this permits intervention in a management and treatment perspective. The intervention can be nutritional, can include the use of dietary supplements, use of specific nutraceuticals, and can include, of course, other conventional and alternative treatments and management of health care.

The database so gathered, while primarily phenotypic in its laboratory analytical sense and its patient descriptive sense, is also predictive for the most part of the genotype of the individual animals or groups of animals in the population, because the canine genome has changed very little over the last hundred years, and so the majority of the canine genome is identical between dog breeds and individual dogs. Differences in phenotype (physical appearance and size and weight, for example) within dog breeds constitutes a very small genetic variation, less than 1%, within the overall genome. Predicting genotype and phenotype with these comprehensive and cumulative laboratory test panels permits a novel approach to intervening in the management and treatment of canine disease and disorders and also in the maintenance of canine health and longevity.

The comprehensive cumulative database developed allows one to look at very early subtle changes that are consistent within individuals or groups of related individual dogs, or animals within a related group so that one can predict disease sooner, make interventions that are less expensive, less invasive, and more effective, and thereby reverse the process before it becomes more serious clinically.

One of the most effective and least invasive or harmful ways to intervene in promoting animal health and longevity is to utilize dietary management. Specifically, wholesome foods are the key to a balanced functioning immune system and the resistance to disease. Given the tight database that is developed by this approach, extraneous noise in the results of comprehensive laboratory analyses is minimized. One can take the findings then for individual animals or groups of animals having cumulative laboratory evidence of trends or drift from the normal ranges and design specific dietary interventions that rebalance the system and promote immunological function and resistance to disease.

This method of identifying what changes can be made in dietary components or supplements does not depend on a single point in time of an individual pet or other animal data, but in fact the key is developing a cumulative comprehensive database over time for normal animals in a like-group location or activity level, as well as specific animals within the group in order to determine what trends are evidenced over time and thereby use the trend to give a more solid determination of what changes are to be made in nutritional requirements or nutritional supplements or other interventions.

### Example 9

### Thyroid Function

A specific example is the diagnostic test panel for thyroid function which depends upon the comprehensive diagnostic test panel and then more specific tests focused on the thyroid, including molecular-based testing and genomic mapping.

Thyroid disease is the most common endocrine dysfunction in companion animals. Thyroid hormone plays a role in metabolism, growth and maturation of the skeletal system, growth and maturation of the central nervous system, and temperature regulation. Early detection of thyroid imbalance allows one to intervene, specifically with nutritional support, and managing individual foodstuffs and supplements that optimizes thyroid function before the disease progresses to the stage where thyroid hormone supplement becomes an essential component of the management and treatment. For instance, food supplements containing kelp, iodine and the minerals from green leafy vegetables are helpful in enhancing thyroid function. Soybean-derived foods and certain other vegetables, by contrast, tend to inhibit thyroid function, as can the protein quality and content in the diet.

In addition to the physiological roles that thyroid hormone plays, it is also recognized that thyroid function plays a role in behavior. Social interaction of the animal with its caregivers is the key to having an individual animal become a successful companion animal member of a household. If the animal has an undesirable behavior or social bad habits, very likely the animal is isolated, stressed, and may be treated unkindly and even ostracized by some family members, so that the animal may eventually be given up or even sent to a pound or shelter and be killed. If kept by the family, the animal may undergo significant stress which contributes to immune suppression and lack of well-being and thus further promotes the abnormal behavior. Thus, it is important to detect thyroid imbalances early in the animal's life.

Thus, the comprehensive individual and group databases of the present invention are used to assess overall health, and specifically thyroid function. By using this database and identifying animals that have very early subtle changes in laboratory analytes shown by their individual or cumulative data drift from the expected normal parameters, one can intervene before the abnormal behavior becomes unbearable for the family caregivers. Yet further, one can intervene before the thyroid imbalance leads to other health concerns, such as poor metabolism.

As it is well known that specific breeds that are used for performance events can have quite different basal thyroid metabolism, for example sighthounds and other coursing breeds vs. toy breeds or working breeds, it is important in the cumulative database to determine these characteristics by comprehensive profiling of this group as a whole, so that the data for individual animals could be compared to the group. Values for this specific functional group by breed are then compared to the entire database for the canine as a species and specific trends over time are developed relating to age and to environmental influences. Once the specific determinants of the individuals and the group that they belong to have been made, the trends that have been identified are used to modify and intervene to promote health and longevity, specifically again with modifications to dietary components or supplements as well as other changes in lifestyle, including exercise, group housing, individual housing and parameters that would promote wellness and longevity.

### Example 10

### Management of Health Using Nutrition

There is a need in the animal health industry for more tightly controlled and designed nutritional supplements and nutraceuticals (i.e., treats) for companion animals such as dogs and cats in order to balance their overall nutritional requirements and thereby improve their immunologic function, health, resistance to disease, and longevity. This needs to be accomplished by taking into account the specific descriptors of the animal such as the breed, age, sex, weight, lifestyle, geographic location, and particular breed function or performance type. These breed and specific animal descriptors need to be linked to some kind of laboratory and general animal health and medical history information.

These nutritional supplements, foodstuffs and/or treats may have a wide variation in terms of flavor, size, and/or shape. For example, a packet in flake form that is sprinkled on top of food; a granola bar; a biscuit; freeze-dried food; or a chewable training treat. Yet further, the nutritional supplement and/or treat may be in a variety of formats that are user-friendly and encourage the owner to purchase it for the benefit of the pet and the pet to eat it.

This example is directed at combining the health information and medical history, including laboratory data which can be simple or comprehensive and cumulative, in order to select a specific nutritional supplement, nutraceutical, treat and/or foodstuff that can correct any imbalances noted in the medical and laboratory health information. This database to help with nutritional management also includes genetic information whenever it is available and combines all of this into a general profile of the specific characteristics of that animal or the animal group that it belongs to. This profile may include retrospective information, current information, and prospective information. At least one of or all of these may be used to determine what the appropriate nutritional management measurements should be.

Implementation of this example is effected by defining which formula of nutritional supplement may be added to the basic food that is appropriate for the animal. Alternatively, the implementation is more complex, where highly, tightly evolved, comprehensive, cumulative database for that particular animal is used to determine which treat is available.

In one implementation of this system, for example, the pet owner goes to a grocery store or a large pet food supply distributing store and selects the appropriate nutritional supplements, nutraceuticals, treat formulations, or foodstuffs for that animal. The store provides the array of supplements and foodstuffs to select from. It has a computer-generated memory database for that specific pet. The information is given a code based on the owner's name, etc., and other information. The owner has a specific number for that, punches their own special confidential number into the computer. The computer then indicates which of the appropriate nutritional supplements, nutraceuticals, treat formulations, or foodstuffs can be selected. The purchaser then goes to the store shelf and picks the one that is appropriate for them. Thus, the owner can take the basic basal food that the animal needs and adds to that the appropriate supplement customized and unique for that individual.

Specific basal diets already exists in the animal/pet food industry—for example, foods for puppies, for adults, for geriatrics, for animals with specific disease states like kidney disease or liver disease or obesity or skin problems or a variety of options. This example builds on this.

As an example, the information in the computerized stored database in the specific store or by identification card is put into the machine which the owner keeps. The card contains all the medical history and also indicates which treat, by number or color code or specific description matches and can be added to the appropriate basal food. Thus, the food purchased is an added nutritional supplement or treat to add to the basic food that has also been determined to be appropriate for that pet.

This is a simple, easily-adapted, commercially viable program to optimize the health and well-being and longevity of companion animals. Specifically, this program is developed as a matching system whereby the information provided selects the appropriate basal diet and then matches that with the specific supplement that is needed for the individual animal or family or group.

The computer terminal user database at the point of sale can be connected to an offsite computer storage database where more sophisticated information is available and stored and would process it there in order to give the remote user, within a few minutes, the appropriate formula that is necessary. This operates in a manner similar to an automated teller machine system at a bank, where there is a central computer that stores all of the database specific and unique to that individual with a pass code that is necessary.

The technology of the example can be applied to veterinary clinics and larger veterinary hospitals, where the veterinarian can be the professional individual inputting the information to select the appropriate nutraceutical or treat for the pet. In this case, one can envision that the veterinarian may be using more therapeutic prescribed supplements to balance the health of the animal, as opposed to more general supplements that might be available, for example at a grocery store or a specialty pet supply outlet.

This example can be extended further, where the owner or the veterinary clinic or other health professional can actually connect through the Internet with a wholesaler or a manufacturer of the food or specific nutritional supplement desired and order it that way for direct delivery.

Other information at point of sale that can be provided may include such customized packaging or labeling of the product can include photographs, specific descriptors, as well as name of the individual animal, that may come out as a preprinted label that could be put on the specific chosen supplement or treat, and also health care advice and other printed material specific for the maintenance and well-being of that pet, including such things as preventive dentistry, annual wellness exams. All of that can be tied together so that when the person accesses this informational base to obtain the specific nutritional supplement required, that all kinds of additional information on a positive sense to provide the owner with a user-friendly synopsis of health would also be available. In addition to this information being available in hardcopy form at the point of sale, it can also be made electronically available through the Internet or some other access code system or communications system.

This system uses pre-selected and pre-prepared formulas to match with the specific needs of the individual companion animal, family, or animal group. The advantage of this system is that these pre-prepared various nutritional supplement products may be made under the tightest control at a manufacturing plant, rather than just being mixed together at the point-of-sale site, where errors could occur and quality control would not be monitored properly.

Many other examples of the invention exist, each differing from others in matters of detail only. The invention is to be determined solely by the following claims.

### Example 11

### Modulation of Disease

It is also envisioned that genotypic data can be used to identify animals that are susceptible to a disease. Once the genetic predictors of disease susceptibility are determined, then a nutritional regimen is established to modulate the disease. It is contemplated that the modulation of the disease results is inhibition and/or prevention of the disease.

The nutritional regimen uses pre-selected and pre-prepared formulas of food to match the specific needs of the individual companion animal, family, or animal group. Nutritional supplements and/or treats may also be used either in combination with the pre-selected and pre-prepared food formula or alone. Nutritional supplements that are used may include vitamins, anti-oxidants, nutraceuticals, etc. It is envisioned that the nutritional supplement and/or treat may be in a variety of formats that are user-friendly and would of course encourage the owner to purchase it for the benefit of the pet and the pet to eat it.

The method of determining a nutritional regimen may be implemented using any of the databases and systems described herein and incorporated into this Example by reference. For example, the pet owner goes to a grocery store or a large pet food supply distributing store and selects the appropriate nutritional supplement for that animal. The store provides the array of supplements to select from. It has a computer-generated memory database for that specific pet. The information can be given a code based on the owner's name, etc., and other information. The owner can have a specific number for that, punches their own special confidential number into the computer. The computer then indicates which of the appropriate nutritional supplements, nutraceuticals, treat formulations can be selected. The purchaser then goes to the store shelf and picks the one that is appropriate for them. Thus, the owner can take the basic basal food that the animal needs and adds to that the appropriate supplement customized and unique for that individual.

Yet further, it is envisioned that a database is not required to determine a nutritional regimen. For example, a single DNA test is employed that determines a specific DNA marker and/or markers that are predictors of disease susceptibility. Once a DNA marker has been identified, a nutritional regimen is determined that intervenes and modulates the development of the disease. Thus, it is envisioned that a DNA test can determine disease susceptibility and the disease can be prevented and/or inhibited by implementing a nutritional regimen.

Although the present invention and its advantages have been described in detail, it should be understood that various changes, substitutions and alterations can be made herein without departing from the spirit and scope of the invention as defined by the appended sentences. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein can be utilized according to the present invention. Accordingly, the appended sentences are intended to include within their scope such processes, machines, manufacture, compositions of matter, means, methods, or steps.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Dodds, W. J. Adv. Vet. Med. 41:715-732, 1999.
Canine Health Conference 1997, National Parent Club of American Kennel Club/AKC Canine Health Foundation. Can. Pract. 23(1):1-56, 1998.
Ru, G. et al., Vet. J. 156:31-39, 1998.
Glickman, L. et al. J. Am. Anim. Hosp. Assoc. 33:197-204, 1997.
Patronek, G, et al., J. Gerontol. 52A:B171-178, 1997.
Dodds, W. J. Can. Pract. 22 (1): 18-19, 1997.
Hancock, W. W. Am. J. Pathol. 148: 681-684, 1996.
Dodds, W. J. Adv. Vet. Sci. Comp. Med. 39: 29-96, 1995.
Happ, G. M. Adv. Vet. Sci. Comp. Med. 39: 97-139, 1995.
Merrill, J. E., Jonakait, A. G. M. FASEB J. 9: 611-618, 1195.
Cavallo, M. G., et al., Clin. Exp. Immunol. 1:1-7, 1994.
Patronek, G. J., Glickman, L. T. Vet. Forum 11(8); 66-67, 1994.
Jackwood, M. W. J. Am. Vet. Med. Assoc. 204: 1603-1605, 1994.
Dodds, W. J., et al., Vet. Pract. STAFF 5(4-5): 1, 14-17; 21-23, 1993.
Stefanon, G., et al., Can. Pract. 18(3); 15-23, 1993.
Dodds, W. J. Vet. Pract. STAFF 4(1-3): 8-10, 1, 26-31; 35-37, 1992.
Dodds, W. J. Vet. Pract. STAFF 4(5); 19-21, 1992.
Brooks, M. B., et al., J. Am. Vet. Med. Assoc. 200:1123-1127, 1992.
Dodds, W. J. Dog World 77(4): 36-40, 1992.
Dodds, W. J. Dog World 77(5): 44-48, 1992.
Dodds, W. J. Dog World 77(10):40-42, 1992.
Goetzl, E. J., Sreedharan, S. P. FASEB J. 6: 2646-2652, 1992.
Elmslie, R. E., et al., J. Vet. Int. Med. 5: 283-293, 1991.
Raymond, S. L., et al., J. Am. Vet. Med. Assoc. 197:1342-1346, 1990.
Patterson, D. F., et al., J. Am. Vet. Med. Assoc. 193: 1131-1144, 1988.
Jolly, R. D., et al., Adv. Vet. Sci. Comp. Med. 25: 245-276, 1981.

1. A method of managing the health care and well-being of a non-livestock animal comprising the steps of:
a) obtaining a database relating to at least one of:
i. species of the animal,
ii. a selected group of the species;
b) obtaining data relating to the animal, the data including laboratory test data relating to the animal;
c) relating the database of a) with the data of b) by a computer; and
d) determining, based on c), a regimen for the management of the animal.

2. A method of managing the nutrition of a non-livestock animal comprising the steps of:
a) obtaining a database relating to at least one of:
i. species of the animal,
ii. a selected group of the species;
b) obtaining data relating to the animal, the data including laboratory test data relating to the animal;
c) relating the database of a) with the data of b) by a computer; and
d) determining, based on c), a nutritional regimen for the management of the animal.

3. A method of managing the health of a non-livestock animal comprising the steps of:
a) obtaining a database relating to at least one of:
i. species of the animal,
ii. a selected group of the species;
b) obtaining data relating to the animal, the data including diagnostic laboratory test data relating to the animal;
c) relating the database of a) with the data of b) by a computer; and
d) determining, based on c), a therapeutic intervention or maintenance for the management of the animal.

4. The method of claim 1, wherein further comprising obtaining genetic profile data relating the animal.

5. The method of claim 1, wherein the species and animal are canine.

6. The method of claim 1, wherein the species and animal are feline.

7. The method of claim 1, wherein the database of the selected group of the species is at least on of breed, age, sex, size, weight, performance use, or geographical location.

8. The method of claim 2, wherein the nutritional regimen is selected from the group consisting of foodstuffs, treats, drinks, nutritional supplements, holistic treatments and exercise.

9. The method of claim 3, wherein the therapeutic intervention or maintenance is selected from the group consisting of drugs, nutraceuticals, nutritional supplements, holistic treatments and exercise.

10. The method of claim 1 wherein the diagnostic laboratory test data is a comprehensive general health profile and selectively at least one selected diagnostic profile for a selected subject.

11. The method of claim 1 wherein the computer uses at least one of an expert system or interrelationship program or network for determining database and data relationships.

12. The method of claim 1 including the step of reporting the determination on a communications network including the Internet.

13. The method of claim 1 including the step of reporting the determination on a communications network including the Internet, and obtaining payment for the report through the Internet.

14. A method of modulating disease in a non-livestock animal comprising the steps of:
a) obtaining a database relating to at least one of:
i. a species of the animal
ii. a selected group of the species;
b) obtaining genotypic data relating to the animal, the genotypic data includes genetic profile data relating to DNA markers associated with the disease.
c) relating the database of a) with the data of b) by a computer; and
d) determining, based on c), a nutritional regimen to modulate the disease.

15. The method of claim 14, wherein modulating is inhibition of the disease.

16. The method of claim 14, wherein modulating is prevention of the disease.

17. A method of modulating a disease in a non-livestock animal comprising the steps of:
a) performing a DNA test to determine a known DNA marker associated with a disease;
b) obtaining the data from the DNA test; and
c) determining based on b), a nutritional regimen to modulate the disease.
